(19) United States
(12) Patent Application Publication
(10) Pub. No.: US 2024/0368208 A1
(43) Pub. Date: Nov. 7, 2024

(54) PROCESS FOR PREPARING DIOSMIN AND FLAVONOID FRACTION

(71) Applicant: LES LABORATOIRES SERVIER, SURESNES CEDEX (FR)

(72) Inventors: Ozkan SARI, Aubervilliers (FR); Bruno SCHIAVI, Gruchet le Valasse (FR); Stéphane LAVAL, DIJON (FR); Corentin RASSON, WAVRE (BE)

(21) Appl. No.: 18/574,801

(22) PCT Filed: Jul. 5, 2022
(86) PCT No.: PCT/EP2022/068596

§ 371 Date: Dec. 28, 2023

(30) Foreign Application Priority Data
Jul. 6, 2021 (EP) 21305932.2

(51) Int. Cl. C07H 17/07

(52) U.S. Cl. CPC C07H 17/07

(57) ABSTRACT

Process for the preparation of diosmin and flavonoid fraction by direct oxidation of hesperidin.

The present invention relates to a process for the preparation of diosmin.

Diosmin is the compound of the formula (I):

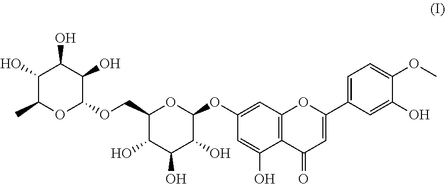

Diosmin is synthesized by oxidation of hesperidin. Hesperidin is the compound of the formula (11):

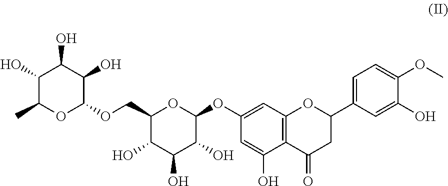

Hesperidin is obtained from natural substances (small oranges). The diversity of the small oranges used produces hesperidins of unequal purity, containing other flavonoids with varying contents.

For example, hesperidin may contain up to 4% isonaringine, which is converted by oxidation into isorhoifoline.

Therefore, diosmin generally contains other flavonoids, some of which result from the oxidation of flavonoids contained in the original hesperidin, while others are by-products of the reaction.

The specifications required by the European Pharmacopoeia for diosmin are as follows:

| | Diosmin specifications |
|---|---|
| Substances | (European Pharmacopoeia) |
| Diosmin | 90.0 to 102.0% |
| Hesperidin | <4.0% |
| Diosmetin | <2.0% |
| Isorhoifolin | <3.0% |
| Linarin | <3.0% |
| 6-Iododiosmin | <0.6% |

Pharmacopoeia Diosmin refers to diosmin that meets the above specifications.

Diosmin is also the predominant component of the micronized purified flavonoid fraction, or MPFF (Daflon®).

Flavonoid fraction refers to an active ingredient comprising around 90% diosmin, in admixture with hesperidin, diosmetin, linarin and isorhoifolin.

The flavonoid fraction according to the present invention preferably comprises 86.0 to 95.0% diosmin, 2.5 to 6.0% hesperidin, 0.2 to 2% diosmetin, 0.8 to 3.5% linarin and 0.8 to 3.5% isorhoifolin.

Additionally, the flavonoid fraction according to the present invention may contain other hesperidin-derived flavonoids with a total content not exceeding 1.0%.

Pharmacopoeia Diosmin and the micronized purified flavonoid fraction are used in the treatment of venous diseases, such as chronic venous insufficiency or hemorrhoidal diseases.

Given their pharmaceutical value, it is paramount to obtain them with excellent yield and high purity, even from hesperidin with a purity of 90-95%.

In particular, it is paramount that the diosmin obtained, whether in the form of Pharmacopoeia Diosmin or flavonoid fraction, contains less than 0.6% 6-iododiosmin.

Processes for the preparation of diosmin from hesperidin have been described in literature.

Patent applications FR2311028 and WO2016124585 describe how diosmin is obtained by acetylation of hesperidin, oxidation of acetylated hesperidin into acetylated diosmin followed by deacetylation.

This is not an ideal process, as the yield is only 65% and 77% respectively. Moreover, it comprises several steps.

Patent applications EP3321273 and FR2782518 describe how diosmin is obtained by oxidation of hesperidin with iodine in a single step, either in an amide solvent (EP3321273) or in pyridine (FR2782518).

However, using iodine in stoichiometric quantities is problematic on an industrial scale.

The problem of the present invention was to produce Pharmacopoeia Diosmin or the flavonoid fraction from hesperidin in a single step before isolation (without going through the step of hesperidin acetylation and deacetylation of the acetylated diosmin), with excellent yield, while minimizing the 6-iododiosmin content in the product obtained.

More specifically, the present invention relates to a process for preparing Pharmacopoeia Diosmin or flavonoid fraction by oxidation of hesperidin into diosmin via an iodine-donating oxidizing couple, at a temperature of 80 to 120° C. in a mixture of polar aprotic solvent and acetic acid, followed by isolation by added water, filtration, rinsing and drying.

According to one embodiment of the present invention, diosmin is obtained in the form of Pharmacopoeia Diosmin.

According to another embodiment, diosmin is obtained in the form of a flavonoid fraction comprising from 86.0 to 95.0% diosmin, from 2.5 to 6.0% hesperidin, from 0.2 to 2% diosmetin, from 0.8 to 3.5% linarin and from 0.8 to 3.5% isorhoifolin.

Additionally, the flavonoid fraction according to the present invention may contain other hesperidin-derived flavonoids with a total content not exceeding 1.0%.

The iodine-donating oxidizing couple is preferably selected from NaI/H₂O₂, KI/H₂O₂, TBAI/H₂O₂ and NaI/I₂/H₂O₂.

The amount of iodine-donating NaI, KI or TBAI is preferably 0.4 to 0.8 molar equivalents relative to the hesperidin involved.

The amount of hydrogen peroxide is preferably 0.9 to 1.1 molar equivalent relative to the hesperidin employed.

Where NaI/I₂/H₂O₂ is used, the NaI/I₂ molar ratio is preferably approximately 9/1.

Examples of polar aprotic solvents that can be used in the process according to the invention include dimethyl sulfoxide, N-methylpyrrolidone and dimethylacetamide.

The preferred polar aprotic solvent is dimethyl sulfoxide.

Where the polar aprotic solvent is dimethyl sulfoxide, S-methylated impurities may be formed in the process. It is important to minimize the content of these impurities.

According to one embodiment of the present invention, the crude diosmin obtained is purified by a base/acid treatment: passage in a water solution in the presence of a base such as sodium hydroxide, then precipitation by salification with an acid such as sulfuric acid, filtration, rinsing and drying.

According to another embodiment of the present invention, crude diosmin is first reslurried in an organic solvent such as dimethyl sulfoxide, N-butylpyrrolidone, pyridine, sulfolane, dimethyl carbonate or propylene carbonate, or in a mixture of organic solvent and water such as a mixture of pyridine and water, then filtered and rinsed with water, before undergoing abase/acid treatment. According to another embodiment of the present invention, the diosmin obtained is purified by a base/acid treatment in the presence of hydrogen peroxide.

The following examples illustrate the invention.

The purity of the hesperidin used in the examples was 93.6%. The use of a high-purity hesperidin makes it possible to obtain Pharmacopoeia Diosmin.

### ABBREVIATIONS

DMSO dimethyl sulfoxide
eq. molar equivalents (relative to hesperidin)
HPLC High Performance Liquid Chromatography
m/m ratio expressed as mass/mass
NBP N-Butyl-Pyrrolidone
ND Not detected
TBAI Tetrabutyl ammonium iodide
rpm revolutions per minute
vol volume equivalent (expressed in L/kg of flavonoid employed)

### EXAMPLE 1: CRUDE DIOSMIN

Into a 6 L reactor, pour in hesperidin (800 g, 1.31 mol, 1.0 eq.) and sodium iodide (123.7 g, 0.82 mol, 0.63 eq.).

Then add DMSO (3.1 vol. 2.48 L) and acetic acid (0.5 vol, 0.40 L).

Stir at 250 rpm (anchor) then heat to 97° C. (T %).

At 97° C., add 3.5M 11202 aqueous solution (367 mL, 0.98 eq.).

After pouring, the heterogeneous reaction medium is left to stir at 97° C. for 15 min, then brought back to 25° C.

At 25° C., water (1.5 vol, i.e. 1.2 L) is added (exotherm of 12° C.), then the medium is filtered through a sintered glass filter with pore size 3.

The cake is rinsed with water (2×2.5 vol, i.e. 2×2.0 L).

The resulting cake is dewatered on a filter. The crude diosmin thus obtained can be taken directly to the next stage.

Alternatively, it can be isolated and dried in a ventilated oven at 80° C. overnight.

Diosmin is then obtained with a yield of 94% and a 6-iodo-diosmin content of 0.52%.

### EXAMPLE 2: BASE/ACID TREATMENT AND MICRONIZATION

Transfer the undried crude diosmin obtained in Example 1 to a 6 L reactor.

Add water (5.0 vol relative to the hesperidin used in Example 1. i.e. 4.0 L) and sodium hydroxide (611 g, 4.58 mol, 3.5 eq.).

Stir at 25° C. until completely dissolved (usually 1 h).

Clarify the medium on a Clarcel Pad®, then transfer the filtrate into a 6 L reactor.

Cool the solution to 10° C., then slowly add 37% sulfuric acid (typically 611 g, 2.30 mol) without exceeding 13° C. (Tₘₐₛₛ) until pH=3 is obtained.

Stir for 45 minutes at 10° C. to finalize precipitation.

Filter the medium through a sintered glass filter with pore size 3.

Rinse the cake with water (2×2.5 vol. i.e. 2×2.0 L).

The product is dried overnight in a ventilated oven at 80° C. The resulting product can be put through a reprocessing step in the presence of hydrogen peroxide.

Alternatively, the solid obtained can be micronized.

Diosmin is then obtained in the form of a micronized flavonoid fraction with 92% yield from hesperidin and a 6-iodo-diosmin content of 0.5%.

| | Percentage in the |
|---|---|
| Substances | Example 2 product |
| Diosmin | 87.8% |
| 6-Iododiosmin | 0.5% |

### EXAMPLE 3: BASE/ACID REPROCESSING IN THE PRESENCE OF HYDROGEN PEROXIDE

Base/acid reprocessing in the presence of hydrogen peroxide removes any S-methylated impurities.

Pour the diosmin obtained prior to micronization in Example 2 (10.0 g, 16.4 mmol) into a 250 mL Erlenmeyer flask.

Then add water (5.0 vol. 50 mL) and sodium hydroxide (7.67 g, 3.5 eq.).

Stir until completely dissolved.

Add 35% hydrogen peroxide (0.1 or 0.2 eq.) in one portion, then stir at room temperature for 30 min to 3 hrs.

Cool to 10° C., then add sulfuric acid (7.40 g, 1.7 eq.) to pH=3.

Filter the medium through a sintered glass filter with pore size 3.

The cake is rinsed with water (2×2.0 vol, i.e. 2×10 mL).

The solid is then dried overnight in a ventilated oven at 80° C.

Diosmin is obtained in the form of a flavonoid fraction.

| | 0.1 eq. H₂O₂ | 0.2 eq. H₂O₂ |
|---|---|---|
| Substances | 30 mn | 3 h |
| Diosmin | 88.1% | 88.6% |
| 6-Iododiosmin | 0.4% | 0.4% |

### EXAMPLE 4: PYRIDINE/WATER RESLURRYING FOLLOWED BY BASE/ACID TREATMENT

The isolated and dried crude diosmin obtained in Example 1 was reslurried in the binary system pyridine/H₂O 6/1 (volume ratio), at 25° C., for 90 min. This reslurrying reduces the 6-iodo-diosmin content and removes any S-methylated impurities.

Transfer the crude diosmin (100 g, dry weight, 164.3 mmol) obtained in Example 1 into a 1 L reactor.

Add water (1.0 vol, or 100 mL) and pyridine (6 vol, or 600 mL).

Stir at 25° C. for 90 minutes.

Filter medium through sintered glass filter with pore size 3.

Rinse the cake with water (2×2.5 vol, i.e. 2×250 mL).

Dewater the solid on a filter and transfer the product without further drying to a IL reactor.

Add water (5.0 vol. 500 mL) and sodium hydroxide (76.7 g, 575.1 mol, 3.5 eq.).

Stir at 25° C. until completely dissolved (usually 1 h).

Clarify the medium on a Clarcel Pad®, then transfer the filtrate into a 1 L reactor.

Cool the solution to 10° C., then slowly add 37% sulfuric acid (typically 76.7 g, 289.3 mmol) without exceeding 13° C. (Tₘₐₛₛ) until pH=3 is obtained.

Stir for 45 minutes at 10° C. to finalize precipitation.

Filter the medium through a sintered glass filter with pore size 3.

Rinse the cake with water (2×2.5 vol, i.e. 2×250 mL).

The product is dried overnight in a ventilated oven at 80° C.

Diosmin is obtained in the form of a micronized flavonoid fraction with a purity greater than 88%, a yield of 88% from hesperidin and a 6-iodo-diosmin content of 0.4%.

Substances

| | |
|---|---|
| Diosmin | 88.4% |
| 6-Iododiosmin | 0.4% |
| S-methylated impurities | Not detected |

### EXAMPLE 5: RESLURRYING IN OTHER SOLVENTS

The crude diosmin used was obtained under the conditions of Example 1.

### DMSO Reslurrying:

Pour diosmin (5.0 g) and DMSO (3 vol) into a 100 mL tricol, then heat at 80° C.: until completely solubilized.

Allow to cool to 25° C., then add water (3 vol). Filter through sintered glass tilter with pore size 3, rinse the cake with water (2×2.5 vol) then dry overnight at 80° C. in a ventilated oven.

Diosmin is obtained in the form of a flavonoid fraction (4.9 g).

### Sulfolane Reslurrying:

Pour diosmin (5.0 g) and sulfolane (7 vol) into a 100 mL tricol, then stir at room temperature for 1 h.

Filter through sintered glass filter with pore size 3. Rinse the cake with water (2×2.5 vol) then dry overnight at 80° C. in a ventilated oven.

Diosmin is obtained in the form of a flavonoid fraction (5.0 g).

### Dimethyl Carbonate Reslurrying:

Pour diosmin (5.0 g) and dimethyl carbonate (7 vol) into a 100 mL tricol, then stir at room temperature for 1 h.

Filter through sintered glass filter with pore size 3. Rinse the cake with water (2×2.5 vol) then dry for 2 days at 50° C. in a ventilated oven.

Diosmin is obtained in the form of a flavonoid fraction (4.9 g).

### Propylene Carbonate Reslurrying:

Pour diosmin (5.0 g) and propylene carbonate (7 vol) into a 100 mL tricol, then stir at room temperature for 1 h.

Filter through sintered glass filter with pore size 3. Rinse the cake with water (2×2.5 vol) then dry for 2 days at 0° C. in a ventilated oven.

Diosmin is obtained in the form of a flavonoid fraction (4.8 g).

### NBP Reslurrying:

Pour diosmin (5.0 g) and N-butylpyrrolidone (7 vol) into a 100 mL tricol, then stir at room temperature for 1 h.

Filter through sintered glass filter with pore size 3. Rinse the cake with water (2×2.5 vol) then dry overnight at 80° C. in a ventilated oven.

Diosmin is obtained in the form of a flavonoid fraction (4.8 g).

| | Crude diosmin | Purification by reslurrying | | | | |
|---|---|---|---|---|---|---|
| | Before | | | Dimethyl | Propylene | |
| Substances | reslurrying | DMSO | Sulfolane | carbonate | carbonate | NBP |
| Diosmin | 88.2% | 86.8% | 86.9% | 86.9% | 86.9% | 87.3% |
| 6-Iododiosmin | 0.5% | 0.5% | 0.5% | 0.5% | 0.5% | 0.4% |
| S-methylated | <0.1% | ND | ND | ND | ND | ND |
| impurities | | | | | | |

1-12. (canceled)

13. A process for the preparation of diosmin by oxidation of hesperidin via an iodine-donating oxidizing couple, at a temperature of 80 to 120° C., in a mixture of polar aprotic solvent and acetic acid, followed by isolation by added water, filtration, rinsing and drying.

14. The process according to claim 13, wherein the diosmin obtained is in the form of Pharmacopoeia Diosmin, comprising from 90.0 to 102.0% diosmin, less than 4.0% hesperidin, less than 2.0% diosmetin, less than 3.0% isorhoifolin, less than 3.0% linarin and less than 0.6% 6-iododiosmin.

15. The process according to claim 13, wherein the diosmin obtained is in the form of a flavonoid fraction comprising from 86.0 to 95.0% diosmin, from 2.5 to 6.0% hesperidin, from 0.2 to 2% diosmetin, from 0.8 to 3.5% linarin and from 0.8 to 3.5% isorhoifolin.

16. The process according to claim 13, wherein the diosmin obtained contains less than 0.6% 6-iododiosmin.

17. The process according to claim 15, wherein diosmin obtained contains less than 0.6% 6-iododiosmin.

18. The process according to claim 13, wherein the iodine-donating oxidizing couple is selected from NaI/H₂O₂, KI/H₂O₂, TBAI/H₂O₂ and NaI/I₂/H₂O₂.

19. The process according to claim 18, wherein the iodine-donating oxidizing couple is NaI/H₂O₂.

20. The process according to claim 19, wherein the amount of NaI is from 0.4 to 0.8 molar equivalent with respect to the hesperidin employed.

21. The process according to claim 18, wherein the amount of hydrogen peroxide is from 0.9 to 1.1 molar equivalents relative to the hesperidin employed.

22. The process according to claim 13, wherein the polar aprotic solvent is dimethyl sulfoxide.

23. The process according to claim 13, wherein the diosmin obtained is purified by a base/acid treatment.

24. The process according to claim 13, wherein the crude diosmin obtained is first reslurried in an organic solvent, including dimethyl sulfoxide, N-butylpyrrolidone, pyridine, sulfolane, dimethyl carbonate and propylene carbonate, or in a mixture of organic solvent and water, including a mixture of pyridine and water, then filtered and rinsed with water, before undergoing a base/acid treatment.

25. The process according to claim 13, wherein the diosmin obtained is purified by a base/acid treatment in the presence of hydrogen peroxide.
